(19) United States
(12) Patent Application Publication
(10) Pub. No.: US 2012/0225132 A1
(43) Pub. Date: Sep. 6, 2012

(54) Methods for Increasing the Kinetic Activity of Alcohol, Water and Other Liquids, so as to Render the Liquids More Capable of Enhancing the Alternative Cellular Energy Pathway in the Prevention and Therapy of Diseases

(76) Inventor: William John Martin, South Pasadena, CA (US)

(21) Appl. No.: 13/040,262

(22) Filed: Mar. 3, 2011

(51) Int. Cl. A61K 33/00; G01N 21/77; A61P 17/02; A61K 31/045

(52) U.S. Cl. 424/600; 514/724; 436/164

(57) ABSTRACT

The kinetic energy of liquids can be increased by placing the liquid in a cobalt blue glass container, which is placed in an area exposed to sunlight. This method can be used in combination with other methods known to activate the kinetic energy of a liquid, as assessed by the neutral red kinetic assay. Infrared emitting ceramic pebbles can similarly be used to activate alcohol. Activated alcohol is more effective than unprocessed alcohol were used with neutral red dye and illuminated with ultraviolet light as a means of enhancing the alternative cellular energy (ACE) pathway in the therapy of diseases.

### CROSS REFERENCE TO RELATED APPLICATIONS CO-PENDING PATENT APPLICATION

Methods for Detection of Ultraviolet Light Reactive Alternative Cellular Energy Pigments (ACE-pigments) William John Martin Submitted Dec. 24, 2007. Publication number 20090163831

Method of assessing and of activating the alternative cellular energy (ACE) pathway in the Therapy of Diseases. William John Martin Submitted Jan. 16, 2008. Publication number 20090181467

Enerceutical mediated activation of the alternative cellular energy (ACE) pathway in the therapy of diseases. Submitted May 8, 2008. Publication number 20090280193

Enerceutical activation of the alternative cellular energy (ACE) pathway in therapy of diseases. Submitted Feb. 11, 2009. Publication number 20090202442 Method of using the body's alternative cellular energy pigments (ACE-pigments) in the therapy of diseases Submitted Feb. 20, 2009. Publication number 20100215763

Urine as a source of alternative cellular energy pigments (ACE-pigments) in the assessment and therapy of diseases. Submitted Mar. 5, 2009. Publication number 20100196297

Diagnostic value of systemic ACE pathway activation in the detection by fluorescence of localized pathological lesions. Submitted Jul. 26, 2010. Publication number 20100291000

Enerceutical mediated activation of the alternative cellular energy (ACE) pathway in the therapy of diseases. Submitted July 2010.

Energy Charged Liquids to Enhance Enerceutical Activation of the Alternative Cellular Energy (ACE) Pathway in the Therapy of Diseases. Submitted Dec. 17, 2010. application Ser. No. 12/972,344

Energy Charged Alcoholic Beverages for Enhancing the Alternative Cellular Energy Pathway in the Prevention and Therapy of Diseases. application Ser. No. 13/016,948 Submitted Jan. 28, 2011.

Methods for Detecting and Monitoring the Activity of Energized Water and Other Liquids Useful for Enhancing the Alternative Cellular Energy Pathway in the Prevention and Therapy of Diseases. Submitted February 2011

Methods for Increasing the Kinetic Activity of Alcohol, Water and Other Liquids, so as to Render the Liquids More Useful in Enhancing the Alternative Cellular Energy Pathway in the Prevention and Therapy of Diseases. Submitted February 2011

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION:

In recently submitted co-pending patent applications, which are incorporated by reference herein, I disclosed that the passage of electrolytically derived Water Gas (otherwise known as Brown's Gas) into alcohol (200 proof absolute ethanol) greatly enhances the ability of the alcohol to react with neutral red dye and with alternative cellular energy (ACE) pigments (defined in the co-pending patent applications). When a small number (usually around 10) of neutral red dye particles (obtained directly from a commercially available neutral red dye powder e.g. Dudley Corp. N.J.), are gently sprinkled onto a plastic dish, which contains Water Gas “charged” alcohol; and the solution is illuminated with visible and/or UV light, there is rapid and vigorous dissolving of most of the material within the neutral red dyes particles. This occurs in a directional manner forming long, narrow red streams of dissolved neutral red. Equally impressive, fine particles, breaking away from the larger particles of neutral red dye, which remain non-dissolved in the alcohol solution, undergo continuing to-and-fro movements, with apparent attractive forces rapidly alternating with repulsive forces. The linearity of the dissolving neutral red and the dynamic movements of non-dissolved particles occur in regular (non-charged) absolute alcohol, but are greatly heightened by Water Gas charging of the alcohol. The use of charged alcohol also significantly enhances the intensity of the orange fluorescence of the neutral red solution, when compared with dye dissolved in non-charged alcohol. As the alcohol (both charged and non-charged) finally evaporates, the precipitating neutral red dye assumes attractive, banded circular patterns, as if being influenced by an interactive energy field. The patterns are more striking using charged alcohol. The charged alcohol also shows greater interaction than does non-charged alcohol with ACE pigments collected from the saliva and/or perspiration of virus infected patients. When used therapeutically, the combination of neutral red with charged alcohol is decidedly more effective in enhancing the ACE pathway in energy deficient individuals, when compared with, still quite impressive, benefits obtained using untreated (non-charged) alcohol.

The linear dissolving pattern of neutral red dye observed in alcohol solutions, was also observed in various alcoholic beverages, especially when they were charged with Water Gas. This extraordinary pattern is not ordinarily seen when neutral red dye is added to ordinary (non-charged) water in similar plastic dishes. Instead, slowly dissolving neutral red particles become gradually surrounded by the dissolved dye in essentially concentric discs of red dye. An exception is seen with some plastic drinking glasses and some plastic covered paper cups. In these containers, there is an obvious attraction between the plastic material and the neutral red particles, leading to radial movements of the particles towards the interface between the surface of the water and the plastic container. (These types of containers are, therefore, unsuitable for used in the experiments described in this application). I have commonly used small (1.5″) square individual polycarbonate dishes and various multi-well tissue culture dishes. The dishes are generally observed using a low power, dissecting microscope with spacing between the light and the dish to help reduce heat transfer. In some experiments light emitting diode (LED) illumination was used to exclude heat, as opposed to light, as the cause of particle movements.

An important finding was observed when neutral red dye particles, are gently sprinkled (scattered) onto a plastic dish of distilled water, and the dish is placed in close proximity to a dish of charged alcohol with moving neutral red dye particles. Instead of the particles in the water remaining essentially stationary and only slowly dissolving, several of the particles will begin to move throughout the water. Moreover, if additional neutral red dye is added, the fresh particles will show more linear dissolving patterns with movements of the remaining non-dissolved particles. The energy transference effect is best seen when the dishes are in direct contact or comprise adjacent wells within the same multi-well dish. Physical contact is not absolutely necessary, however, and the effect has been seen between separated dishes. I, thereby, discovered that the altered dissolving pattern of neutral red dye in ordinary water potentially provided a simple assay for detecting a radiating energy coming from the well containing the charged alcohol and neutral red particles. The assay also provides a means of detecting the water and alcohol energizing capacities of various additional forms of energy, in addition to that provided by Water Gas. For convenience, this assay will be subsequently referred to as the “NR-Kinetic Assay.”

The more recent patent application explored various alternative methods to the use of Water Gas to enhance the kinetic activity of water, alcohol and alcoholic beverages. Effective methods included the use of magnetic, electromagnetic, electrostatic and sound energies. Application of these methods, either individually or more impressively in combination and with the inclusion of added Water Gas, markedly enhanced the capacity of alcohol plus dissolved neutral red dye to activate the ACE pathway in an autistic patient, with an impaired ACE pathway. Individual methods and especially the combination of the methods, also enhance the interaction of water and of alcoholic beverages with neutral red dye, as assessed by the NR-Kinetic assay system.

The present patent application extends the previous findings with an additional method of adding energy (as assessed by the NR-Kinetic assay) to water, alcohol and alcoholic beverages. The method comprises using cobalt blue colored containers for the various liquids. This approach was prompted by a publication by Augustus James Pleasonton. His book entitled “On the Influence of the Blue Color of the Sky in Developing Animal and Vegetable Life” was read before the Philadelphia for Promoting Agriculture in September 1871 with a follow up book report in 1876 entitled “The Influence of the Blue Ray of the Sunlight and of the Blue Color of the Sky.” Basically, he postulated that sunlight absorbed by cobalt blue colored glass was converted into another energy form, which had growth promoting and healing benefits on plants, animals and humans. Although, he presented factual information, his thesis was discarded as being illogical. As disclosed in the present patent application, cobalt blue colored containers can significantly increase the reactivity of neutral red with water, alcohol and alcoholic beverages, as assessed by the NR-Kinetic assay. More importantly, this method has added to the effectiveness of the procedure to enhance the ACE pathway, which comprises the ultraviolet light illumination of neutral red dye in processed alcohol solution.

The research has given added credibility to the ACE providing qualities of water, alcohol and alcoholic beverages. Of widespread practical importance is that ordinary beverages can be processed in a number of ways with additive effects on their ability to enhance the ACE pathway of plants, animals and humans. While, the present focus of ACE pathway activation is on the suppression of stealth adapted virus infections, the approach has clear application to other infectious illnesses, including herpes virus infections. Moreover, beneficial effects from enhancing the ACE pathway are expected in a wide range of diseases caused by inadequate cardiac, vascular, respiratory and/or metabolic functions. Other major application of promoting the ACE pathway is in wound repair, tissue regeneration, anti-ageing and even as a means to cosmetically improve appearance. The inclusion of cobalt blue containers in the processing of ACE enhancing liquids is a valuable addition to the methods disclosed in previous patent applications.

### BRIEF SUMMARY OF THE INVENTION

Easily discernable increased activity, as determined by the NR-Kinetic assay, was demonstrated in liquids within cobalt blue glass containers, compared to similar liquids contained in plain glass containers, when the containers were exposed to sunlight for varying amounts of time. The effect was particularly striking using alcohol (200 proof ethanol). Moreover, the use of the cobalt blue colored container was clearly additive to previously shown combined effects on alcohol achieved by i) bubbling Water Gas through the liquids; ii) using a rotating magnet; iii) exposure to sound energy, including sound of approximately 500 Hz; iv) exposure to a strong electrostatic field, as provided by a Van de Graaff generator; and v) exposure to electromagnetic radiation from an electrical power cord. Cobalt blue glass and plastic containers are, thereby, capable of enhancing the kinetic energy property of liquids and the capacity of the liquids to activate the ACE pathway.

The beneficial effect of sunlight exposed cobalt blue on the clinical efficacy of ethanol samples treated additively by all of the other methods listed above was evaluated by the mother of a 15 year old child with autism.

### BRIEF DESCRIPTION OF THE DRAWINGS

Not Applicable and none included

### DETAILED DESCRIPTION OF THE INVENTION

The NR-Kinetic Assay has previously been employed in the direct assessment of various kinds of processed liquids, including absolute alcohol and alcoholic beverages. For instance, it was also used to demonstrate the marked effect of simply bubbling Water Gas through absolute alcohol on the ability of the “charged” alcohol to react with neutral red dye. Not only was there more of a direct effect on neutral red added to the Water Gas charged alcohol, such as more intense fluorescence, but the light illuminated charged alcohol solution containing neutral red dye, was clearly able to distantly induce movements of non-dissolved neutral red dye particles placed in an adjacent well containing regular water. In this assay, the light illuminated neutral red dye containing charged alcohol was placed in one of the wells of a tissue culture plate. Adjacent wells contained water plus stationary un-dissolved neutral red dye particles. These wells were observed microscopically to see whether any of the non-dissolved neutral red particles would begin to move, which they did beginning approximately 30-60 seconds later. An additional observation on the adjacent neutral red dye in water wells included the occasional formation of numerous gas bubbles, which might well represent the formation of Water Gas. The energy transference type of assay consistently provided positive results, which were not seen using multiple wells in other culture dishes, which simply had water plus neutral red solutions. The Water Gas charged alcohol was consistently judged to be superior to unprocessed alcohol when tested on a child with autism, using a standardized protocol, described in prior patent applications.

The availability of the NR-Kinetic assay has allowed for the testing of various other suggested methods for energizing water and for pioneering these methods for the purpose of activating alcohol, alcoholic beverages and potentially all other types of liquids. Energy activating activity of water, alcohol and alcoholic beverages was shown using i) rotating bar magnets; ii) computer generated sound frequencies, including 500 Hz; iii) placement within the vicinity of an active electric power cord, emanating an electromagnetic field; and iv) exposure to a strong electrostatic field provided by a battery driven van de Graaff generator. A final procedure applicable to all samples of ethanol for clinical use with neutral red dye and UV illumination is the jolting of the solution for about 20 times, similar to the method referred to as succussion for homeopathic solutions.

I have been aware for several years of the reports of General Pleasonton of the use of blue glass panes in the design of his greenhouse. He alternated blue glass panes with ordinary transparent glass panes and observed much improved growth of his gape vines and other vegetations. He extended his findings to the use of blue glass cover over chicken hatchery and pigpens in controlled studies. Other individuals confirmed these findings and, indeed, it became fashionable to add blue glass to solaria for its supposed human health benefits. The practice was criticized in a Scientific American publication as being illogical and fraudulent and was largely abandoned.

Cobalt is widely used to render glass a deep blue color. I have accumulated various cobalt blue bottles, jars and other containers. It was, therefore, relatively easy to place samples of water, alcohol and alcoholic beverages into cobalt blue glass containers and to use liquids in ordinary glass containers as controls. The containers were exposed to sunlight for varying times ranging from 30 minutes to 8 hours. Samples were taken for analysis using the NR-Kinetic assay. Increasing with time, but certainly seen at 1 hour, the liquids within the cobalt blue containers displayed significantly more kinetic activity than did the corresponding control liquids. The effect was most noticeable using ethanol and further confirmed by the marked increase more yellowish fluorescence under ultraviolet light. Although reduced after being brought indoors, the differences in kinetic activity and in UV fluorescence were still noticeable a day later.

A major question has been whether the various procedures of enhancing the kinetic activity of liquids were each additive or whether some were duplicative. Preference was also to be given to simple and lasting procedures versus complicated and short acting procedures. A standardized sequence of procedures was developed for ethanol. It comprised bubbling of Water Gas into the alcohol, followed by magnetic rotation (now using Jura-Capresso Inc. milk frother machine); exposure to 500 Hz sound and placement over an active power cord. While 30 minutes time periods for each procedure are effective, time periods for an hour were more generally used. One-ounce aliquots of the standardized treated ethanol were sent to the mother of a 15 year old autistic girl. The mother, with input from her child, has become a very reliable monitor of the effectiveness of different ethanol solutions in their capacity to achieve clinical improvement. She had previously evaluated her child's response to each of the aforementioned procedures used individually and concluded that each procedure provided ethanol, which was better than the unprocessed ethanol; (which was still effective). She further confirmed that ethanol processed by using the standardized combined procedures was superior to any samples she had previously tested. She was asked to further expose an aliquot of ethanol, previously subjected to the combined standardized procedures, to the sunlight cobalt blue glass container method. Both my e-mail response and on direct interview, she reported on a significantly improved response of the solution after it had been left for an hour in a cobalt blue glass bottle. The child showed an exceptional initiative in challenging herself with difficult tasks, which she was comfortably completing. When asked why she was doing so well, she immediately responded that it was because of the day's light treatment. Her mother further commented upon her bright eyes making direct contact, clear speech and improved physical agility. Without hesitation, the mother scored the solution as a further improvement on the previously used standardized solutions.

In further confirmation of this conclusion has been the quite remarkable patterns formed by neutral red as it precipitates from evaporating solutions of the now newly re-standardized ethanol solutions. Multiple sharply defined concentric rings form from the solutions, with some of the material being surprisingly poorly re-soluble when additional alcohol is added.

The other major line of inquiry is to show beneficial effects on the ACE pathway of consuming water, which has been placed in cobalt blue colored glass and/or plastic containers and exposing the containers to sunlight. As with the alcohol, it is reasonable to presume that combinations of water processing procedures will prove more effective than using only one procedure. Possibly, only for appearance, some bottled water is currently being sold in blue plastic and glass bottles. The information disclosed in the present patent application provides a rational basis to indicate the benefit of this practice in terms of adding to the kinetic energy of liquids, including drinking water.

Various solutions for possible cosmetic and wound healing benefits are also being prepared by mixing activated water and activated ethanol in various proportions. Using activated solutions, a 10% ethanol in water is proving beneficial as a twice-daily facial wash in a test subject.

In a more recent series of experiments, small baked ceramic pebbles, known to spontaneously emit infrared radiation were added to ethanol for one hour and for longer periods. Interestingly, this simple procedure also enhanced the kinetic activity of the ethanol in the NR-Kinetic assay. The effect was seen at 1 hour and required no more than 4 approximately five mm diameter pebbles per ml. In addition to the rapid formation of long linear streaks of dissolving neutral red dye, multiple, fine concentric rings of precipitated dye were readily seen following the evaporation of the dye in alcohol solution. The ceramic pebbles used in these experiments were produced from silica-based volcanic clay and marketed as a water purifying material. It is anticipated that not all ceramic pebbles will show activity in the NR-Kinetic assay. For those that do, they can be used to prepare alcohol and other liquids, which will be more capable than the corresponding unprocessed control liquid of enhancing the ACE pathway in the prevention and therapy of diseases

The principles, preferred embodiments and modes of operation of the present invention, intended to be protected herein, is not to be construed as limited to the use of either cobalt blue colored containers or infrared emitting ceramic stones as ways of enhancing the energetic activity of liquids, including water and alcohol, intended for use in the activation of the ACE pathway Co-pending patent applications provide several additional examples, including the use of electrolytically generated Water Gas and energy fields of magnetic, sound, electromagnetic and electrostatic activities. The patent application is not restricted to the use of neutral red as the indicator dye in the kinetic assay, since several other dyes can and have been used in similar experiments. Neutral red has the advantage, however, of potentially being used in therapeutic endeavors aimed at activating the ACE pathway. ACE pigments, collected from a patient's saliva or perspiration, are also anticipated as being more active in energized, compared with regular water and alcohol. The term “energized” is also not meant to be exclusive of other relevant terms, which I or other investigators may use in furthering the research described in this application.

Additional modifications of the basic tenets disclosed in the present patent application will readily occur to those skilled in the art and especially upon practicing the currently described methods. Therefore, many variations and changes may be made without departing from the scope and spirit of the invention as encompassed by the appended claims.

1. A method of treating an individual, comprising the use of a liquid solution, which has been processed in such a manner that it will show enhance kinetic linear and to-and-fro oscillatory movement of particles of an added dye, such as neutral red dye, and further comprising the use of the solution in a procedure, which results in local and/or systemic activation of the alternative cellular energy (ACE) pathway of the treated individual.

2. The method of claim 1, in which the solution is an alcohol and the processing technology is the placement of the solution in a blue colored container, preferably containing cobalt as the cause of the blue color, for sufficient duration so as to enhance the kinetic linear and to-and-fro oscillatory movement of particles of an added dye, such as neutral red dye.

3. The method of claim 1, in which the solution is an alcoholic beverage and the processing technology is the placement of the solution in a blue colored container, preferably containing cobalt as the cause of the blue color, for sufficient duration so as to enhance the kinetic linear and to-and-fro oscillatory movement of particles of an added dye, such as neutral red dye.

4. The method of claim 1, in which the solution is water and the processing technology is the placement of the solution in a blue colored container, preferably containing cobalt as the cause of the blue color, for sufficient duration so as to enhance the kinetic linear and to-and-fro oscillatory movement of particles of an added dye, such as neutral red dye.

5. The method of claim 1, in which the solution is alcohol and the processing technology is the placement of infrared emitting ceramic pebbles, or other structures into the solution for sufficient duration so as to enhance the kinetic linear and to-and-fro oscillatory movement of particles of an added dye, such as neutral red dye.

6. The method of claim 1, in which the solution is an alcoholic beverage and the processing technology is the placement of infrared emitting ceramic pebbles, or other structures into the solution for sufficient duration so as to enhance the kinetic linear and to-and-fro oscillatory movement of particles of an added dye, such as neutral red dye.

7. The method of claim 1, in which the solution is water and the processing technology is the placement of infrared emitting ceramic pebbles, or other structures into the solution for sufficient duration so as to enhance the kinetic linear and to-and-fro oscillatory movement of particles of an added dye, such as neutral red dye.

8. A method for testing a procedure to determine whether it can activate alcohol, an alcoholic beverage, water or other liquid, as determined by testing for enhanced reactivity of the liquid with neutral red dye, following the procedure; the enhanced reactivity being the faster speed of formation and the longer lengths of linear streaks forming from dissolving neutral red dye, together with more noticeable to-and-fro oscillatory movement of non-dissolved particles of neutral red and with more clearly defined banding patterns of precipitating neutral red dye forming as the solution fully evaporates.

9. The method of claim 1, in which the procedure comprises the use of the processed liquid, preferably an alcohol, with the addition of a small quantity (approximately 0.1-1.0 mg/ml) of added neutral red dye, which the solution being either directly applied to an area of the body, or placed within a container which is laid onto the body; followed by ultraviolet light illumination of the solution in such as manner, which results in local and/or systemic activation of the alternative cellular energy (ACE) pathway of the treated individual.

10. The method of claim 1, in which the procedure comprises the use of the processed liquid, preferably water, which is ingested in a sufficient quantity result in the local and/or systemic activation of the alternative cellular energy (ACE) pathway of the treated individual.

11. The method of claim 1, in which the activation of the alternative cellular energy (ACE) pathway of the treated individual is intended as a means of tissue regeneration and/or wound repair.

12. The method of claim 1, in which the activation of the alternative cellular energy (ACE) pathway of the treated individual is intended as a means of enhancing physical appearance through an improvement in complexion.
